(19) United States
(12) Patent Application Publication
(10) Pub. No.: US 2006/0271193 A1
(43) Pub. Date: Nov. 30, 2006

(54) Device for connecting a longitudinal member to a bone

(76) Inventors: Stephan Hartmann, Solothurn (CH); Armin Studer, Langendorf (CH)

Correspondence Address: JONES DAY, 222 EAST 41ST STREET, NEW YORK, NY, 10017-6702, US

(21) Appl. No.: 11/373,780

(22) Filed: Mar. 9, 2006

Related U.S. Application Data
(63) Continuation of application No. PCT/CH03/00643, filed on Sep. 26, 2003.

(51) Int. Cl. A61F 2/44

(52) U.S. Cl. 623/17.11

(57) ABSTRACT

A device to join a longitudinal support (3) with a bone, in particular with a body of the vertebra, comprising a bone anchoring element (1) that can be fixed on a bone, a connecting element (9) provided on the rear end (5) of the bone anchoring element (1) with a channel axis (27) passing through the connecting element (9) transversely to the central axis (2) to accept a longitudinal support (3), tightening means (13) which can be connected with the free end (21) of the connecting element (9) and are suitable for the fixing of a longitudinal support (3) introduced into the channel (25), and a tilting element (8) provided in the channel (25), said tilting element being rotatable about both the central axis (2) and the axis (7) of rotation that is transverse to the axis (7) of the channel.

### CROSS-REFERENCE TO RELATED APPLICATIONS

This application is a continuation of International Application No. PCT/CH2003/000643, filed Sep. 26, 2003, the entirety of which is incorporated by reference herein.

### FIELD OF THE INVENTION

The invention concerns a device to join a longitudinal support with a bone, in particular with a body of the vertebra.

### BACKGROUND OF THE INVENTION

A device with a fastening element to fix a pedicle screw or a pedicle hook on a longitudinal support is known from EP 0 572 790. In the case of this known device the fastening element is firmly joined with the pedicle screw or the pedicle hook and comprises a tilting element that can rotate about an axis of rotation that is perpendicular to both the central axis of the fastening element and the longitudinal axis of the longitudinal support. The longitudinal support lies on the top surface of the tilting element, that curves coaxially with the longitudinal axis of the longitudinal support, and by means of a tightening element is pressed against the tilting element. With its bottom surface, curved coaxially with the longitudinal axis of the longitudinal support, the tilting element lies in the fastening element on the complementarily formed bottom of the channel. A disadvantage of this known device is, that the bottom surface of the tilting element with the bottom form a force-locking joint only, so that in the case of greater torques, acting about the axis of rotation, an undesirable rotation may occur between the longitudinal support and the pedicle screw or pedicle hook.

The object of the invention is therefore to produce a device that allows a form-locked locking of a rotating joint against the relative rotation between the longitudinal support and the bone anchoring element.

### SUMMARY OF THE INVENTION

This objective is achieved by the invention by a device to join a longitudinal support with a bone, in particular with a body of the vertebra.

The advantages achieved by the invention are essentially that by virtue of the device according to the invention a form-locked locking of a rotating joint can be produced between a longitudinal support and a bone anchoring element.

In a preferred embodiment the second contact surface K2, that forms the bottom of the channel, has three-dimensional macroscopic structures. The connecting element with the macroscopic structures is preferably made from a material that is harder than the tilting element, for example from a titanium alloy or a Ti/Al/niobium alloy. The softer tilting element is preferably made from pure titanium. By virtue of this it will be achieved, that when the tightening means is tightened, the macroscopic structures on the contact surface K2 of the harder connecting element are pressed into the softer tilting element, thus producing a form-locking connection between the two parts. Thus a torque, exerted on the longitudinal support by the levering forces of the bone anchoring element, cannot lead to a displacement of the two contact surfaces K1, K2 relative to one another, so that the angle between the central axis of the bone anchoring element and the longitudinal axis of the longitudinal support, set on the longitudinal support during the fixing of the bone anchoring element, will not change even in the case of high torques, so that a good stabilization of, for example, adjacent bodies of the vertebra can be produced.

In another embodiment both contact surfaces K1, K2 have three-dimensional macroscopic structures, wherein the three-dimensional macroscopic structures are configured preferably by complementary serrations with the serrations extending parallel to the axis of rotation. This will result in the fact that the form-locking connection between the connecting part and the tilting element can be achieved without any deformation of one of the two parts.

On the other hand the three-dimensional structures may have pyramid-shaped or cone-shaped teeth, or truncated pyramid-shaped or truncated cone-shaped teeth, while in this case, provided both contact surfaces K1, K2 have macroscopic structures, the structures can be constructed in a complementary manner.

The height of the three-dimensional macroscopic structures, measured at right angles to the contact surfaces K1, K2, is preferably between 0.1-5.0 mm.

In yet another embodiment the tilting element comprises a top concave surface, that can radially abut against a longitudinal support and has a groove extending transversely to the axis of rotation and the central axis. Centrally, between the two ends of the tilting element, which intersect the longitudinal axis of the longitudinal support, the groove preferably has a depression. This will bring with it the advantage, that when the tightening means is tightened the longitudinal support is pressed into and deformed in the depression, so that the locking between the longitudinal support and the connecting element will be intensified.

In a further embodiment the tilting element comprises on each of its lateral surfaces, which are perpendicular to the axis of rotation, a groove with the shape of a circular arc and concentric with the axis of rotation, said grooves being enclosed at that ends of the tilting element which intersect the longitudinal axis of the longitudinal support. Furthermore, on the connecting element two pins are provided which engage the grooves. Due to this the connecting element and the tilting element are loosely held together, so that during the implantation none of the parts could be lost yet an in-situ alignment of the longitudinal support will not be hindered.

The connecting element and the bone anchoring element are preferably made integral.

### BRIEF DESCRIPTION OF THE DRAWINGS

The invention and developments of the invention are explained in detail in the following based on the partly schematic illustrations of one embodiment:

FIG. 1 is a perspective illustration of an embodiment of the device according to the invention,

FIG. 2 is a side view of the embodiment of the device according to the invention illustrated in FIG. 1, and

FIG. 3 is a front view of the embodiment of the device according to the invention illustrated in FIGS. 1 and 2.

### DETAILED DESCRIPTION OF THE INVENTION

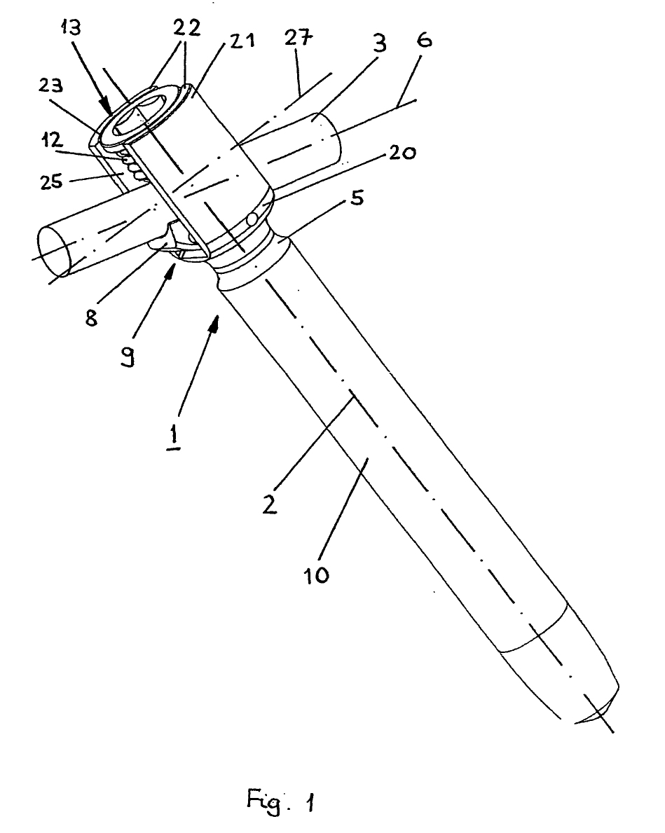

FIGS. 1 to 3 illustrate an embodiment that as a bone anchoring element 1 has a screw shaft 10, coaxial with the central axis 2, to be screwed into a pedicle of a body of the vertebra. The connecting element 9, firmly joined with the rear end 5 of the screw shaft 10, is passed through by a channel 25, having a channel axis 27 transversely to the central axis 2 and having essentially a U-shape. Each of the two legs 22, bordering the channel 25, have a fastened end 20 and a free end 21. The channel axis 27 is perpendicular in this case to the central axis 2. The width of the channel 25 is b, and it is so chosen that the longitudinal support 3 is held by the legs 22 transversely to its longitudinal axis 6. A bore 23 with an inside thread 24 is provided in the connecting element 9 penetrating from the free end 21 of the two legs 22, the axis of the bore being concentric with the central axis 2 and its inside diameter d being greater than the width b of the channel 25. The tightening means 13 is constructed in this case as a tightening screw 12 with a shaft that can be coaxially screwed into the inside thread 24, preferably with a sawtooth-like thread, in the connecting element 9, the tightening screw having a front end 14 and a rear end 15. From the rear end 15 an internal hexagon 16 penetrates into the tightening screw 12 coaxially with the central axis 2 to accept a screwdriving tool. When the tightening screw 12 is tightened, the front end 14 of the tightening screw 12 is pressed against the surface of a longitudinal support 3 placed into the channel 25, so that the longitudinal support will be fixed in the connecting element 9 of the bone anchoring means 1. In the channel 25 at the fastened ends 20 of the two legs 22 the tilting element 8 is provided. The tilting element 8 is mounted in the channel 25 in such a manner, that it can rotate about an axis 7 of rotation that is transverse to the central axis 2 and the channel axis 27. The bottom 26 of the channel bounds the channel 25 in the direction of the rear end 5 of the screw shaft 10, has a concave construction. The bottom 26 of the channel bounds the channel 25 with a circular arc shape when viewed in a cross-section that is perpendicular to the axis 7 of rotation, while the centre of the circular arc is the axis 7 of rotation.

The tilting element 8 comprises two lateral surfaces 37, 38, which are perpendicular to the axis 7 of the rotation, a bottom surface 28 with a convex construction and facing the fastened end 20 of the connecting element 9, and a top surface 29 with a concave construction and facing the free end 21 of the connecting element. At the same time the bottom, convex surface 28 has a construction that is complementary to the bottom 26 of the channel, while the top, concave surface 29 has such a construction, that a groove 36 is formed to accommodate the longitudinal support 3, said groove being parallel to the longitudinal axis 6 of the longitudinal support 3 and having a depression 30 centrally between the two the ends bounding the tilting element 8 transversely to the axis 27 of the channel. A longitudinal support 3, placed into the channel 25, is placed on the top concave surface 29 of the tilting element and together with the tilting element 8 it can rotate about the axis 7 of rotation. When the tightening means 13 is fixed, the longitudinal support 3 is pressed into the groove 36 and the depression 30 and is bent in the region of the depression 30, so that the fixing of the longitudinal support 3 will be intensified.

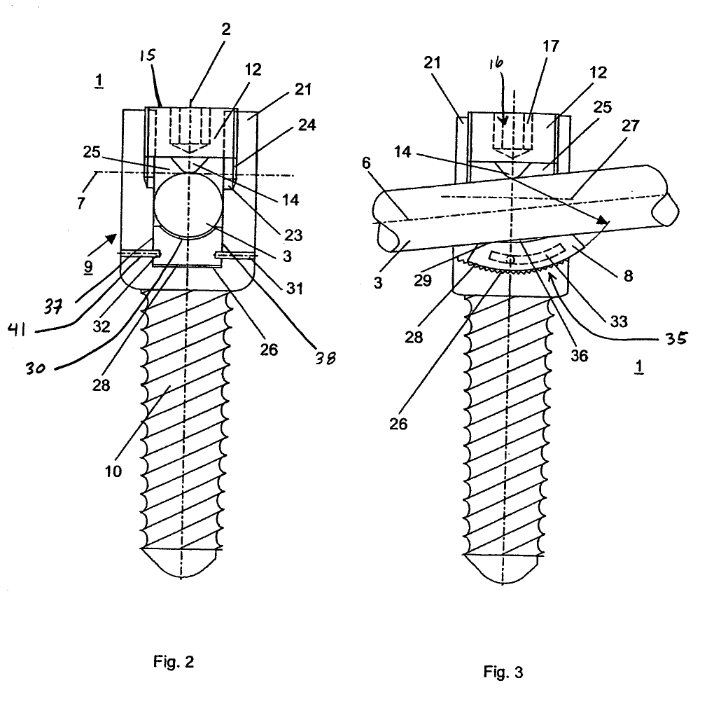

As it is illustrated in FIGS. 2 and 3, at its front end 14 the tightening screw 12 is rounded and can be pressed against the surface of the longitudinal support 3, placed into the channel 25. At its rear end 15 the tightening screw 12 comprises means 17 to accept a screwdriving tool and is screwed into an inside thread 24, said thread provided in a bore 23, penetrating from the free end 21 of the connecting element 9. In the embodiment illustrated here the channel 25 has a U-shaped construction and is open towards the free end 21 of the connecting element 9, so that the bore 23 with the inside thread 24 is also passed through diametrally by the channel 25. Prior to fixing the longitudinal support 3 in the channel 25, the tilting element 8 is displaceably mounted on the bottom 26 of the channel. By virtue of the concave camber of the bottom 25 of the channel, when rotated, the tilting element 8 will rotate about the axis 7 of rotation. The axis 7 of rotation is perpendicular to both the central axis 2 and the channel axis 27 and preferably axially coincides with the contact position between the front end 14 and the surface of the longitudinal support 3 on the central axis 2. On its lateral surfaces 37, 38 the tilting element 8 comprises two grooves 33, which are enclosed towards the two ends of the tilting element 8 and curve concentrically with the axis 7 of rotation on a circular arc between the ends.

Two pins 31, which are pressed into bores 41 in the two legs 22 of the connecting element 9 provided diametrally relative to the central axis 2, engage with their front ends 32 the grooves 33, so that the tilting element 8 is loosely joined with the connecting element 9. The bottom 26 of the channel is provided with macroscopic structures 35, that is configured in this case as a serration and when tightening the tightening screw 12 it can be pressed into the bottom surface 28 of the tilting element 8, so that a form-locking connection can be produced between the connecting element 9 and the tilting element 8.

What is claimed is:

1. A spinal fixation system comprising:
a connecting element having a connecting portion and a bone anchoring portion, wherein the connecting portion has an upper opening, a lower surface, and a channel extending therethrough;
a tightening element insertable through the upper surface and engageable with the connecting portion;
a tilting element disposed in the connecting portion, and having a upper surface and a lower surface, wherein the lower surface of the tilting element is proximate the lower surface of the connection portion; and
a longitudinal member disposed in the channel and proximate the upper surface of the tilting element;
wherein the tilting element is able to rotate relative to the connecting element when the tightening element is not in contact with the longitudinal member; and
wherein the lower surface of the tilting element is configured to securedly engage the lower surface of the connecting portion when the tightening element exerts a downward force on the longitudinal member, thereby fixing the tilting element relative to the connecting portion.

2. The system of claim 1, wherein at least one of the lower surface of the tilting element and the lower surface of the connecting portion has a plurality of projections.

3. The system of claim 2, wherein at least a portion of the projections are serrations.

4. The system of claim 2, wherein at least a portion of the projections are teeth.

5. The system of claim 1, wherein both the lower surface of the tilting element and the lower surface of the connecting portion have a plurality of projections, and wherein the projections are substantially complementary between the surfaces.

6. The system of claim 1, wherein the lower surface of the tilting element as a plurality of projections configured to penetrate the lower surface of the connecting portion.

7. The system of claim 6, wherein lower surface of the connecting portion is comprised of a softer material than the projections.

8. The system of claim 1, wherein the lower surface of the connecting portion has a plurality of projection configured to penetrate the lower surface of the tilting element.

9. The system of claim 8, wherein the lower surface of the tilting element is comprised of a softer material than the projections.

10. The system of claim 1, wherein the lower surface of the tilting element is substantially convex, and the lower surface of the connecting portion is substantially concave.

11. The system of claim 1, wherein the bone anchoring portion is integral with the connecting portion.

12. The system of claim 1, wherein the tightening element is configured to threadedly engage the connecting portion.

13. The system of claim 1, wherein the tilting element further comprises a groove, wherein the connecting portion houses at least one pin engageable with the groove to retain the tilting element within the connecting portion.

14. The system of claim 13, wherein the groove is substantially arc-shaped.

15. A method of securing a spinal fixation system comprising:
(a) providing a spinal fixation system comprising a connecting element having a connecting portion and a bone anchoring portion having a longitudinal axis, a tightening element, and a tilting element disposed in the connecting portion and having a upper surface and a lower surface;
wherein the connecting portion has an upper opening, a lower surface, and a channel extending therethrough;
wherein the lower surface of the tilting element is proximate the lower surface of the connection portion;
(b) inserting at least a portion of the bone anchoring portion into a vertebral body;
(c) inserting a longitudinal element having a longitudinal axis into the channel, such that the longitudinal element is proximate the upper surface of the tilting element;
(d) adjusting the tilting element such that the longitudinal axis of the longitudinal element forms a desired angle with the longitudinal axis of the bone anchoring portion;
(e) manipulating the tightening element, such that the tightening element exerts a downward force on the rod, which in turn exerts a downward force on the tilting member thereby fixing the tilting member relative to the connecting portion.

16. The method of claim 15, wherein step (e) further comprises rotating the tightening element relative to the connecting portion.

17. The method of claim 15, wherein the bone anchoring portion is integral with the connecting portion.

18. The method of claim 15, wherein both the lower surface of the tilting element and the lower surface of the connecting portion have a plurality of projections, and wherein the projections are substantially complementary between the surfaces.

19. The method of claim 15, wherein at least one of the lower surface of the tilting element and the lower surface of the connecting portion has a plurality of projections.
